(19) United States
(12) Patent Application Publication
(10) Pub. No.: US 2012/0136197 A1
(43) Pub. Date: May 31, 2012

(54) HEARING PROSTHESIS HAVING A FLEXIBLE ELONGATE ENERGY TRANSFER MECHANISM

(76) Inventor: Peter B.J. Van Gerwen, Keerbergen (BE)

(21) Appl. No.: 12/957,085

(22) Filed: Nov. 30, 2010

(51) Int. Cl. A61F 11/04; H04R 25/00

(52) U.S. Cl. 600/25

(57) ABSTRACT

Aspects of the present invention are generally directed to using a flexible conductor for transmitting energy to a recipient's cochlea. The flexible conductor may be used to transmit mechanical energy, such as sound vibrations, to the cochlea from an actuator implanted, for example, in a recess in the mastoid bone of the recipient. The flexible conductor may be coupled at its distal end to a component of the recipient's middle or inner ear. In operation, mechanical movement (e.g., vibrations) may be transferred from the actuator to the middle or inner ear of the recipient by the flexible conductor. These vibrations then induce movement of perliymph in the recipient's cochlea to cause, for example, perception of the sound by the recipient.

### BACKGROUND

1. Field of the Invention

The present invention relates generally to hearing prostheses, and more particularly, to hearing prostheses having a flexible elongate energy transfer mechanism.

2. Related Art

Hearing loss, which may be due to many different causes, is generally of two types, conductive or sensorineural. In many people who are profoundly deaf, the reason for their deafness is sensorineural hearing loss. This type of hearing loss is due to the absence, destruction or damage to the hairs in the cochlea which transduce acoustic signals into nerve impulses. Various hearing prostheses have been developed to provide individuals suffering from sensorineural hearing loss with the ability to perceive sound. One type of hearing prosthesis, referred to as a cochlear implant, includes an electrode assembly implanted in the cochlea. Electrical stimulation signals are delivered directly to the auditory nerve via the electrode assembly, thereby inducing a hearing sensation in the implant recipient.

Conductive hearing loss occurs when the normal mechanical pathways which conduct sound to the cochlea are impeded. This problem may arise, for example, as a result of damage to the ossicular chain or ear canal. Individuals suffering from conductive hearing loss frequently retain some form of residual hearing because the hairs in the cochlea are often undamaged. For this reason, individuals who suffer from conductive hearing loss typically are not candidates for a conventional cochlear implant because insertion of the electrode assembly into the cochlea may severely damage or destroy the remaining hairs in the cochlea.

Individuals suffering from conductive hearing loss typically receive an acoustic hearing aid. Hearing aids receive ambient sound, amplify the sound, and direct the amplified sound through the ear canal. The amplified sound reaches the cochlea and causes motion of the cochlea fluid, thereby stimulating the hairs in the cochlea.

Unfortunately, hearing aids do not benefit all individuals suffering from conductive hearing loss. For example, some individuals are prone to chronic inflammation or infection of the ear canal. Other individuals have malformed or absent outer ear and/or ear canals as a result of a birth defect, or as a result of common medical conditions such as Treacher Collins syndrome or Microtia.

Individuals unable to benefit from hearing aids may benefit from implantable hearing prostheses that deliver mechanical energy to the recipient. In one type of implantable hearing prosthesis, an implanted actuator is rigidly connected to the ossicular chain, thereby enabling direct vibration of the ossicular chain to induce an auditory response. In another type of hearing prosthesis, an implanted actuator is rigidly connected to the cochlea and operates by directly vibrating the perilymph in the inner ear. Both of these types of hearing prostheses often require complicated surgery, and they are not well-suited for implantation into growing children because of the rigid connections between the actuator and the ossicular chain and perilymph, respectively.

Another type of hearing prosthesis, referred to as a bone conduction device, such as a Baha®, has an actuator implanted into the skull bone of the recipient. The actuator provides vibrations directly to the recipient's skull bone. These vibrations are conducted by the recipient's bony structure to the inner ear to elicit an auditory response.

### SUMMARY

In one aspect of the invention, there is provided a hearing prosthesis for delivering sound vibrations to a component of a recipient's ear, the hearing prosthesis comprising: an implantable actuator configured to generate the sound vibrations; and a longitudinally-rigid and laterally-flexible elongate conductor having opposing ends adapted to be connected to the actuator and component of the ear, wherein the flexible conductor is configured to transfer the sound vibrations from the actuator to the component of the ear.

In another aspect, there is provided a method comprising: implanting a flexible conductor in a recipient, wherein the flexible conductor is longitudinally rigid and laterally flexible; coupling a first end of the flexible conductor to a component of an ear of the recipient; and coupling a second end of the flexible conductor to an actuator configured to generate sound vibrations representative of an acoustic signal, such that the flexible conductor is configured to transport the sound vibrations from the actuator to the ear component

### BRIEF DESCRIPTION OF THE DRAWINGS

Illustrative embodiments of the present invention are described herein with reference to the accompanying drawings, in which:

FIG. 1A is a partial sectional view of a human skull showing the outer, middle, and inner ear;

FIG. 1B provides a simplified illustration of an implanted hearing prosthesis configured to provide mechanical stimulation to a recipient's inner ear;

FIG. 1C is a simplified view of an implanted hearing prosthesis, in accordance with an embodiment of the present invention;

FIG. 2 illustrates successive lengths of a flexible conductor each having a different acoustic impedance, in accordance with an embodiment of the present invention;

FIG. 3 illustrates a flexible conductor fixed to a structure using a fixation device, in accordance with an embodiment of the present invention;

FIG. 4A illustrates a flexible conductor coupled to a ball-shaped coupling element, in accordance with an embodiment of the present invention;

FIG. 4B illustrates a flexible conductor coupled to a stapes prosthesis, in accordance with an embodiment of the present invention;

FIG. 4C illustrates a flexible conductor coupled to a footplate prosthesis, in accordance with an embodiment of the present invention;

FIG. 4D illustrates a flexible conductor coupled to an ossicular chain prosthesis, in accordance with an embodiment of the present invention;

FIG. 5 is a partial sectional view of a skull showing the ear canal, the cochlea, and an implanted mixed-mode hearing prosthesis, in accordance with an embodiment of the present invention;

FIG. 6 illustrates a simplified flow chart of operations for surgically inserting a flexible conductor within a patient, in accordance with an embodiment of the present invention.

FIG. 7 illustrates an exemplary acoustic transfer function of a stimulation arrangement using a stiff connection between the actuator and a structure in the middle ear or inner ear; and

FIG. 8 illustrates an exemplary transfer function of a stimulation arrangement including a DACS actuator and a flexible sound conductor.

### DETAILED DESCRIPTION

Aspects of the present invention are generally directed to a hearing prosthesis having an implantable mechanical actuator and an elongate flexible conductor that transfers mechanical energy in the form of sound vibrations from the actuator to a component of the recipient's ear. The actuator may be fixedly implanted at any suitable location. The ear component to which the conductor is coupled may be a middle ear component, such as a bone of the ossicular chain, or a component of the inner ear, such as the cochlear promontory, a natural or artificial fenestration of the cochlea, or a semicircular canals.

Characteristics of the conductor such as its material composition, density, dimensions, sheathing, etc., are selected to attain a desired acoustic transfer function. The same or other characteristics of the conductor are selected for one or more lengths of the conductor to attain a sufficient mismatch in acoustic impedance between each such length and the surrounding tissue. This facilitates the efficient transfer of sound vibrations from the actuator to the coupled ear component.

Traditionally, the implanted location and orientation of an actuator depends on the configuration of the actuator and its rigid actuator arm, as well as the location of the ear component that is to be connected to the actuator. In contrast, the actuator and flexible conductor eliminates such dependencies, increasing the viable implant locations for the actuator thereby reducing the likelihood of complications that may occur during implantation due to such restrictions. This, in turn, may reduce the overall surgical time and the amount of time required for the patient to heal. In operation, sound vibrations are transferred from the actuator to the ear component by the flexible conductor. The vibrations induce movement of the perliymph in the cochlea to cause, for example, perception of the sound by the recipient.

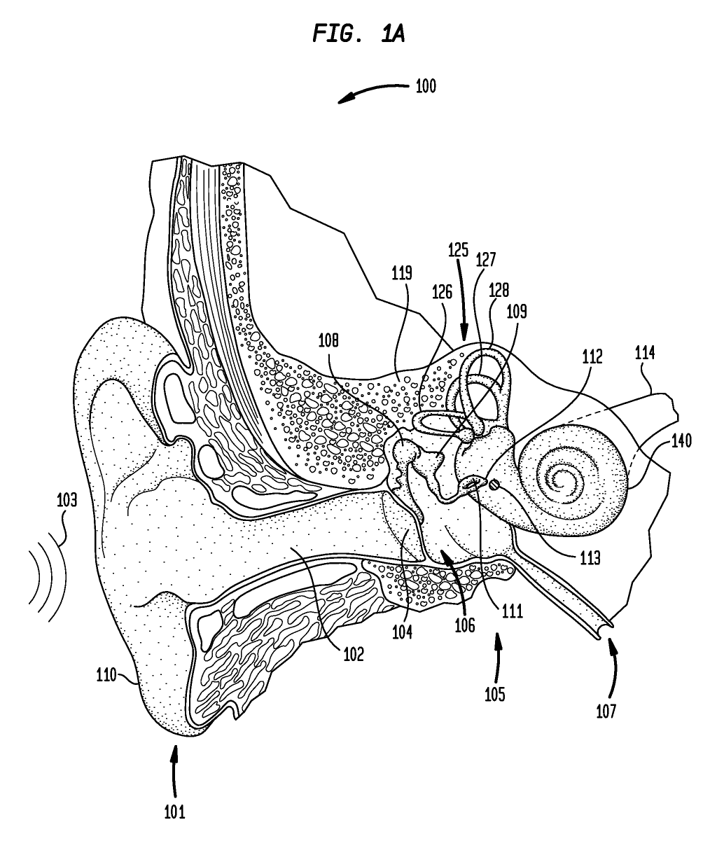

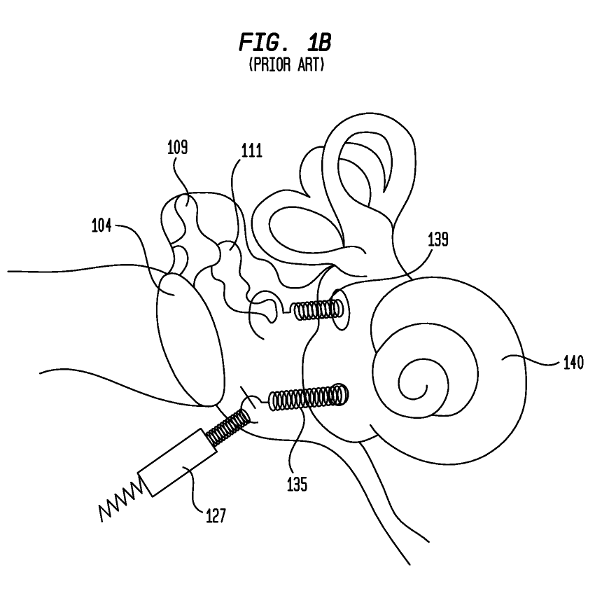

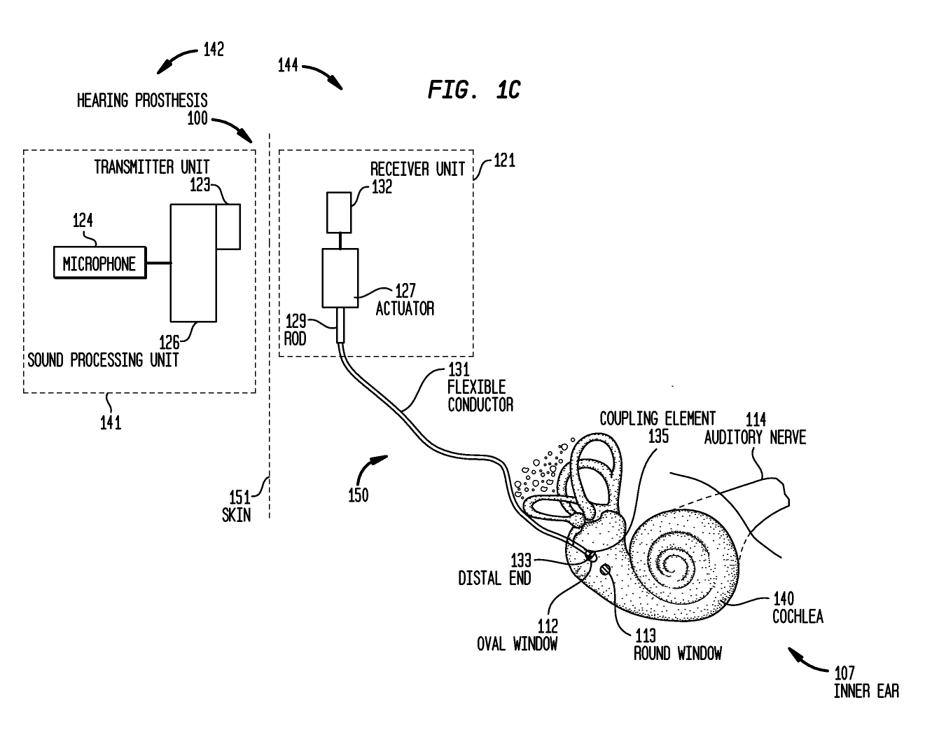

FIG. 1A is a partial sectional view of a human skull showing outer, middle, and inner ear. In a fully functional ear 100, outer ear 101 comprises an auricle 110 and an ear canal 102. Acoustic pressure or sound waves 103 are collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound waves 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Inner ear 107 comprises cochlea 140 and semicircular canals 125. Semicircular canals 125 are three half-circular, interconnected tubes located adjacent cochlea 140. The three canals are the horizontal semicircular canal 126, the posterior semicircular canal 127, and the superior semicircular canal 128.

A system that directly generates mechanical motion of the fluid with a recipient's cochlea is sometimes referred to as a direct acoustic cochlear stimulator (DACS). U.S. Patent Publication No. 2009/0306458 by John Parker et al., entitled “Direct Acoustic Cochlea Stimulator for Round Window Access,” provides a more detailed explanation of an exemplary DAC. A modification of the Parker et al. DACs was proposed by Mojalla, Lenarnz et al. at the 9^{th} Deutsche Fesselschaft für Angiologie (DGA) Jahrestagung.

FIG. 1B provides a simplified illustration of the system proposed by Mojalla et al. The system proposed by Mojallal et. al. is based on the principle of a power-driven stapes prosthesis and is intended for the treatment of severe mixed hearing loss due to advanced otosclerosis. It comprises an implantable electromagnetic transducer (referred to as actuator 127), that is rigidly coupled to the inner ear (e.g., cochlea 140) such that the transducer directly transfers acoustic energy directly to the inner ear. The device of Mojalla et al. is implanted using a specially developed retromeatal microsurgical approach. This approach involves removal of the stapes 111; after which, a coupling element 135 (in this case a conventional stapes prosthesis) is attached to the transducer and placed in the round window 113 to allow direct acoustical coupling to the perilymph of the cochlea 140. In order to restore the natural sound transmission of the ossicular chain, a second stapes prosthesis 139 is placed in parallel to the first one into the oval window 112 and attached to the patient's own incus 109, as in a conventional stapedectomy.

As noted above, in embodiments of the present invention, the location of actuator 127 need not be located a precise distance and position relative to the inner ear of ossicle chain of the recipient. Rather, the actuator may be fixed to the skull (e.g., in the mastoid) at a convenient location. FIG. 1C is a simplified diagram of a mechanical stimulator 100 implanted in a recipient having an outer ear 101, a middle ear 105 and an inner ear 107, such as illustrated in FIG. 1A. For clarity, the surrounding tissues, with the exception of the inner ear, are not shown in FIG. 1B. In the illustrative embodiment, mechanical stimulator 100, sometimes referred to herein as direct mechanical stimulator herein, is a hearing prosthesis which simulates natural hearing by directly generating mechanical motion of the fluid within a recipient's cochlea, thereby activating cochlear hair cells and evoking a hearing percept. For ease of explanation, mechanical stimulator 100 will be referred to hereinafter as hearing prosthesis 100 in describing FIG. 1C.

As illustrated, hearing prosthesis 100 comprises an external component 142 that may be directly or indirectly attached to the body of the recipient, and an internal component 144 which is temporarily or permanently implanted in the recipient. External component 142 comprises one or more sound input elements, such as microphones 124, a sound processing unit 126, a power source (not shown), and an external transmitter unit 123, each of which may be housed within a common housing 141. The external transmitter unit 123 may provide power and stimulation data to internal component 144.

Internal component 144 comprises an internal receiver unit 132 and a stimulation arrangement 150. Internal receiver unit 132 comprises an internal coil (not shown), and preferably, a magnet (also not shown) fixed relative to the internal coil. Internal receiver unit 132 is hermetically sealed within a biocompatible housing 121. The internal coil receives power and stimulation data from the external transmitter unit 121.

Stimulation arrangement 150 comprises an actuator 127, a rod 129, a flexible conductor 131, and a coupling element 135. As illustrated, actuator 127 and rod 129 may be housed in housing 121 with flexible conductor 131 exiting housing 121. Housing 121 may be attached to mastoid bone 119, such as for example, by using screws that may be inserted through openings in the housing 121 and inserted into bone 119. Actuator 127 may be an actuator such as disclosed in International Patent Publication No. WO 2006/058368 entitled “Implantable Actuator for Hearing Aid Applications.”

As illustrated, actuator 127 is connected to rod 129, which is in turn connected to flexible conductor 131. Flexible conductor 131 runs from rod 129, through mastoid bone 119 of the recipient, to its distal end 133 positioned at or in inner ear 107 of the recipient. At distal end 133, flexible conductor 133 is connected to coupling element 135, which couples flexible conductor 133 to oval window 112 of inner ear 107.

In an embodiment, flexible conductor 131 may be a flexible wire that is capable of conducting mechanical vibrations (such as mechanical acoustic vibrations) from actuator 127 and/or rod 129 to oval window 121. Coupling element 135 may be any type of appropriate component, such as, for example, a shoe coupler 135 that may be coupled to oval window 112.

Rod 129 may be fixed to actuator 127 and include a connector for connecting to flexible conductor 131. For example, in an embodiment, rod 129 may include a threaded socket and flexible conductor 131 may include threads on its proximal end to allow flexible conductor 131 to be screwed into and connected to rod 129. In other embodiments, other mechanisms may be used for connecting rod 129 and flexible conductor 131, such as welding the two pieces together or, for example, rod 129 and flexible conductor 131 may be molded together to from a single contiguous component.

As will be discussed in more detail below, coupling flexible conductor 131 to oval 112 window is but one example for providing mechanical stimulation to recipient, and in other embodiments, flexible conductor 131 may be coupled (e.g., using a coupling element) to other regions of middle ear 105 or inner ear 107. For example, in embodiments, flexible conductor 131 may be coupled to round window 113, or a bone of ossicles 106. Or, in yet another embodiment, a fenestration may be created in cochlea 140, and the flexible conductor 131 coupled to the fenestration.

In operation, sounds waves 103 are received by one or more microphones 124, processed by sound processing unit 126, and transmitted by transmitter unit 123 as encoded data signals to internal receiver 132. Internal receiver unit 132 provides the received data signals to actuator 127. Based on these received signals, actuator 127 generates vibrations representative of the sound waves.

Actuator 127 transfers this actuation to rod 129, flexible conductor 131, and coupling element 135, such that they cause mechanical movement of oval window 112. This mechanical movement generates a wave of fluid motion in the perilymph of cochlea 140. Such fluid motion, in turn, activates the hair cells of cochlea 140, which in turn causes appropriate nerve impulses to be generated and transferred through the spiral ganglion cells (not shown) and auditory nerve 114 to the brain (also not shown) where they are perceived as sound.

In an embodiment, flexible conductor 131 may be constructed to have an acoustical impedance mismatch with the surrounding tissues. As used herein, the term tissue refers to any ensemble of cells, such as, muscle, bone, nervous tissue (nerves, the brain, etc.) and connective tissue. This acoustical impedance mismatch may help ensure that the acoustic or vibratory energy is not dampened or absorbed by the surrounding tissues as the energy is transported down the entire length of flexible conductor 131. This may accordingly help ensure that the energy transported by flexible conductor 131 is sufficient to induce an appropriate auditory response in the recipient.

Constructing flexible conductor to have an acoustical mismatch with the surrounding tissues may be accomplished by appropriate selection of material or geometry of flexible conductor 131. For example, flexible conductor 131 may be a wire constructed from titanium and having a fixed diameter of approximately 100 micrometers. In other embodiments, however, flexible conductor 131 may have any diameter or any cross-sectional configuration, such as square, round, oval or irregular. Different diameters and/or geometrical configurations may be appropriate for different implant recipients in helping achieve the desired amount of impedance mismatch.

As noted, to facilitate the efficient transfer of sound vibrations from the actuator to the coupled ear component, characteristics of the flexible conductor are selected to attain a desired acoustic transfer function as well as to ensure there is sufficient acoustic impedance mismatch between the flexible conductor and its surrounding environment. Because the flexible conductor may travel through tissue (e.g., bone, fluids, muscle, etc.), air pockets, etc., the acoustic impedance of the biological environment may vary along the length of the flexible conductor. As such, the characteristics of the flexible conductor may be selected for one or more lengths of the conductor to attain a sufficient mismatch in acoustic impedance between each such length and its immediate environment (e.g., the surrounding tissue).

The acoustic impedance Z₀ of a material is defined by the following formula 1:

Z₀=ρ·c (1),

where Z₀ is the characteristic acoustic impedance of the material, ρ is the density of the medium, and c is the sound speed.

If an acoustic wave in a medium 1 having an acoustic impedance Z₁ encounters a boundary with a medium 2 having an acoustic impedance Z₂, the relation between the amplitudes of the reflected and transmitted waves determine the transmission factor, T, which is defined by the following formula 2:

T=2·Z₁/(Z₁+Z₂) (2)

A perfect match of the acoustic impedances Z₁ and Z₂ results in a transmission factor, T, equal to 1. That means that all of the acoustic energy transmits the boundary. Since the exemplary embodiment, acoustic energy is desired to be kept within medium 1 in order to achieve wave guidance, a mismatch between medium 1 and 2 may be achieved. In an embodiment, the material of medium 1 (e.g., the flexible conductor 131) is chosen so that a transmission of the acoustic wave through the boundary surface of the two media is less than 0.2, where medium 2 may be, for example, the tissue(s) of the recipient.

This explanation is intended to merely show the principle of a mismatch between acoustic impedances, and real world applications may be more complex. For example, the acoustic impedance of a material may depend on other factors besides the type of material used, such as, for example, the acoustic impedance may depend also on the shape of the material (i.e., the flexible sound conductor 131) and the frequency of the sound.

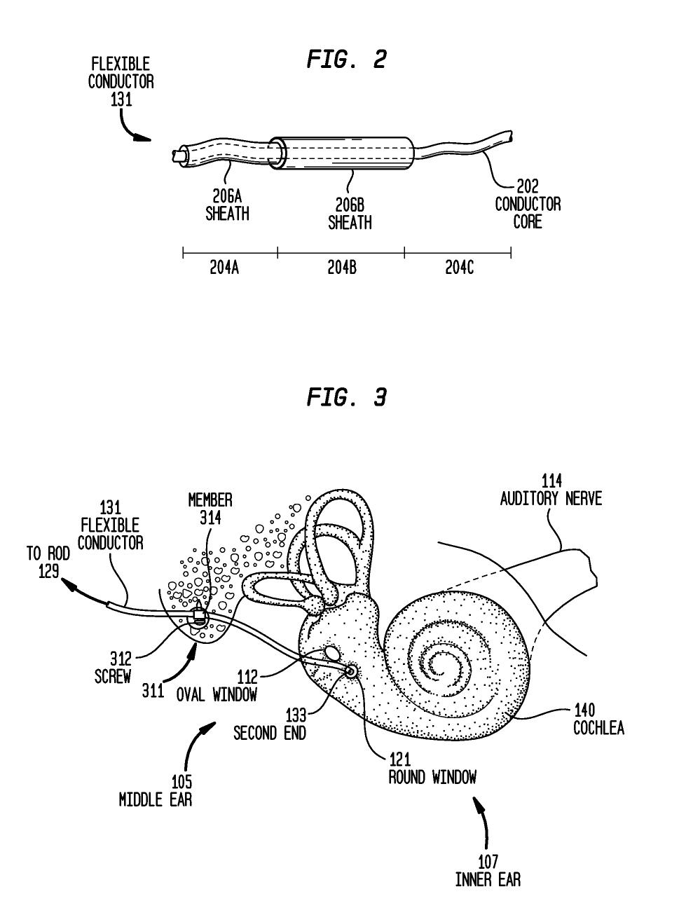

FIG. 2 is a perspective view of an embodiment of flexible conductor 131, showing three lengths 204A, 204B and 204C. Each length 204 has a combination of materials, density, cross-sectional area or volume, sheathing, etc. For example, conductor core 202 in length 204A is formed of material that is different than the material used to form the core in lengths 204B and 204C. Similarly, lengths 204A and 204B have sheaths 206A and 206B, respectively, while length 204C does not have an outer sheath. Further, sheath 206A is formed of a different material and has a different thickness than sheath 206B. Sheath 206A and sheath 206B may be, for example, a coating or sleeve placed around flexible conductor core 202 and may be formed of any appropriate material (e.g., silicone) serving to decouple the flexible connector from the surrounding tissues. For example, each sheath 206 may alter the acoustic impedance of the flexible conductor 131 in the regions where the sheath is located. Thus, different shapes and materials for the sheaths may be used to provide the flexible conductor 131 with different acoustic impedances along its length. For example, a portion of the flexible conductor 131 may be expected to be located next to bony tissue, while another portion may be expected to be located in fluid. Using different sheaths in these different locations may help improve the acoustic impedance mismatch for these different portions.

In an embodiment, flexible conductor 131 may comprise one or more fixation devices for helping stabilize flexible conductor 131. Such a fixation device may be used to attach flexible conductor 131 to stable tissue of the patient, such as to the bone or fibrous tissue of the patient, so as to help reduce or minimize movement of flexible conductor 131 in one or more directions (e.g., prevent or limit movement in a direction perpendicular to the longitudinal length of flexible conductor 131).

FIG. 3 illustrates a close-up perspective view of a flexible conductor 131 comprising a fixation device 311, in accordance with an embodiment. For simplification, ossicles 106 are not shown in FIG. 3. In this example, flexible conductor 131 is coupled to round window 113. As illustrated, fixation device 312 is located along flexible conductor 131 at an intermediary point between rod 129 and the second end 133 of flexible conductor 131. In an embodiment, fixation device 311 may comprise a screw 312 and a member 314 through which screw 312 may be inserted. Member 314 may be configured to acoustically decouple screw 312 from flexible conductor 131 so that, for example, vibrations transported by the flexible conductor are not absorbed or significantly dampened by screw 312. Member 314 may be, for example, a sleeve of a material that absorbs vibrations and may be slid over flexible conductor 131 and positioned at an appropriate intermediate position along flexible conductor 131. Member 314 may have an opening through which screw 312 may be inserted. Screw 312 may then be screwed into the patient's bone, such as, for example, in middle ear 105 or inner ear 107. For example, screw 312 may be inserted into a bone of the ossicular chain, the mastoid bone, the round window, the oval window, the cochlea, an artificial window through custom fenestration, the vestibulum, the promontorium, another bony structure, etc. Once fixed to middle ear 105, fixation device 311 may help stabilize flexible conductor 131 and reduce stresses placed on the coupling between coupling element 135 and inner ear 107 (e.g., round window 113). For example, fixation device 311 may prevent or limit movement of flexible conductor 131 in a direction perpendicular and/or parallel to the longitudinal length of flexible conductor 131. Although FIG. 3 illustrates only a single fixation device, in other embodiments, multiple fixation devices may be used. Additionally, alternative locations along the length of flexible conductor 131 may be used along the length of flexible conductor 131 for position such fixation devices.

As noted above, distal end 133 of flexible conductor 131 may be coupled to inner ear 107 or middle ear 105. For example, in the embodiments of FIGS. 1 and 3, flexible conductor 131 is illustrated as coupled to oval window 112 or round window 113, respectively of inner ear 107. However, in other embodiments, flexible conductor 131 may be coupled to other portions of inner ear 107 or middle ear 105, such as, for example, to one or more semicircular canals 125 or inner ear 107, or one or more of the bones (i.e., oscicles 106) of middle ear 105. Stimulation arrangement 150 may use different types of coupling elements 135 depending on the portion of middle ear 105 or inner ear 107 to be coupled to flexible conductor 131.

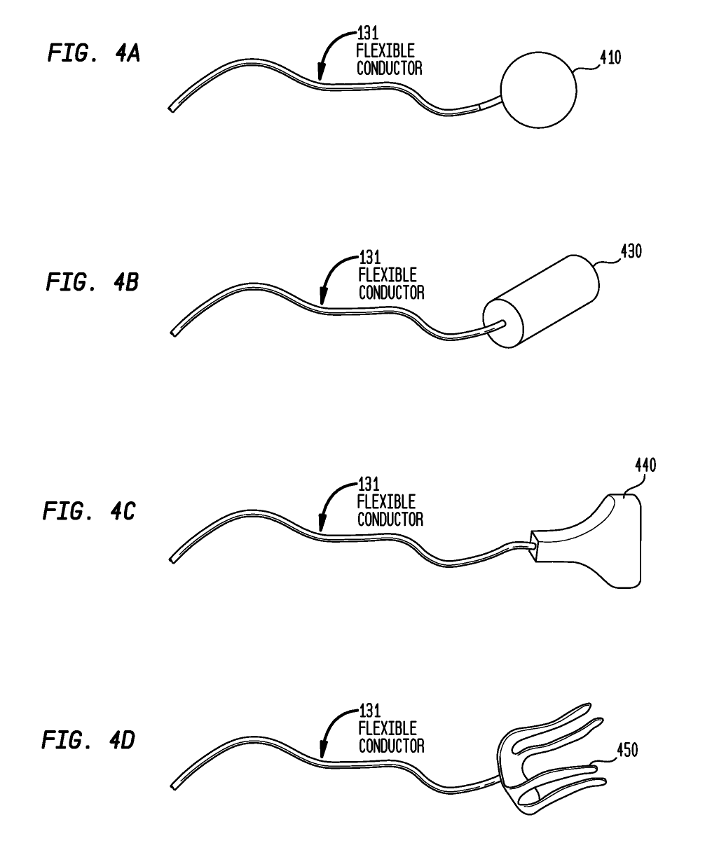

FIGS. 4A-E illustrate various coupling elements that may be used to couple distal end 133 of flexible conductor 131 to the recipient's middle ear 105 or inner ear 107. FIG. 4A illustrates a ball shaped coupling element 410 coupled to the distal end of flexible conductor 131. Ball shaped coupling element 410 may be used to vibrationally couple distal end 133 of flexible conductor 131 to a fenestration leading to the inner ear such as, for example, oval window 112 or round window 113 as discussed above with reference to FIGS. 1 and 3. As used herein, vibrationally coupling the flexible conductor to a fenestration means that the coupling element is configured to transfer vibrations induced in the flexible conductor to the fenestration. For example, as noted above, actuator 127 may induce vibrations in flexible conductor 131 in accordance with received sound. The coupling element may then transfer these vibrations to the fenestration, which in turn causes vibrations in the perliymph, etc., as discussed above, so that the recipient may perceive the received sound.

FIG. 4B illustrates a stapes prosthesis 430 coupled to distal end 133 of flexible conductor 131. As illustrated, in this example, a stapes prosthesis has a cylindrical shape. Stapes prosthesis 430 may be used to vibrationally couple flexible conductor 131 to a fenestration leading to the inner ear such as, for example, oval window 112 or round window 113 as discussed above with reference to FIGS. 1 and 3.

FIG. 4C illustrates a prosthesis 440 adapted to couple distal end 133 of flexible connector 131 to the footplate of stapes 111 to vibrationally couple energy generated by an actuator to the stapes footplate. As illustrated, prosthesis 440 has a wider base on its distal end and gradually becomes narrower towards its proximal end.

FIG. 4D illustrates an ossicular chain prosthesis 450 coupled to distal end 133 of flexible conductor 131. Ossicular chain prostheses 450 may be used to couple flexible conductor 131 to a bone or bone(s) of ossicles to acoustically or vibrationally couple energy generated by an actuator to the ossicular chain. As illustrated ossicular chain prosthesis 340 has four prongs on its distal end that are connected to a base on its proximal end, where the base is connected to distal end 133 of flexible conductor 131.

It should be noted that the exemplary coupling elements of FIGS. 4A-4E are exemplary only, and that in other embodiments other shapes may be used, for example, to clip onto a bone of the ossicular chain, connect through the oval or round window or through a cochleostomy, etc.

As noted above, using a flexible conductor may make it easier to surgically implant the hearing prosthesis into a recipient over other mechanical stimulators or bone conduction devices. Further, because the conductor is flexible, positioning of the actuator is not critical, unlike prior prostheses where the actuator is coupled to structures in the middle or inner ear through a direct, rigid coupling connection. The flexibility of the conductor and the wide array of options available for positioning the actuator reduce the complexity of the surgical procedure for implantation. Further, the flexibility of the conductor enables implantation of the hearing prosthesis described herein into a growing child without significant risk that growth of the child will result in breaking the flexible conductor or injury to the child, or both. Rather, as the child grows, the flexible conductor may flex due to the child's growth rather than break or cause other components (e.g., screws) to become damaged or dislodged.

A hearing prosthesis using a flexible conductor such as described above may also be used in a mixed-mode device. As used herein, the term mixed-mode device refers to a device capable of providing two or more modes of stimulation. Exemplary such modes of stimulation include electrical, mechanical (e.g., acoustic, electro-mechanical, etc.), and optical stimulation. For example, exemplary mixed mode devices might include a hearing prosthesis that can provide both acoustic stimulation (such, as with a hearing aid) in combination with mechanical stimulation, such as discussed above with reference to FIG. 3.

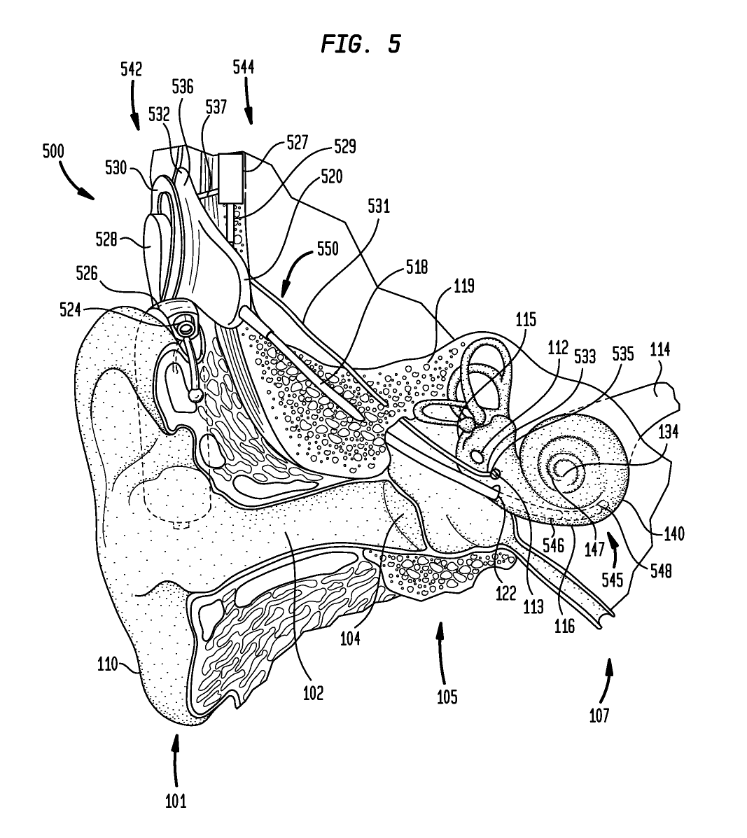

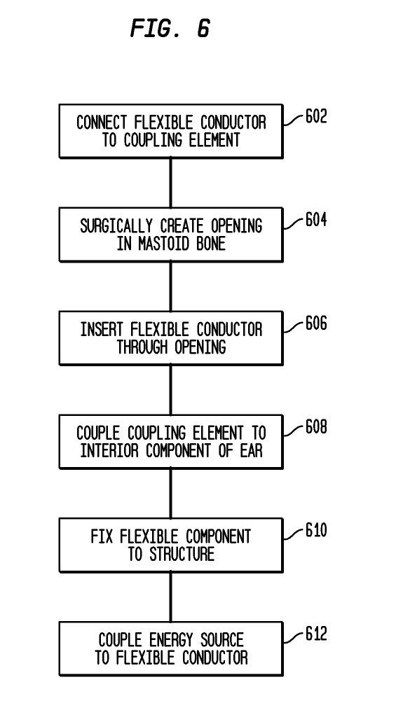

FIG. 5 is a perspective view of a mixed-mode hearing prosthesis 500 capable of providing both mechanical and electrical stimulation. As illustrated, hearing prosthesis 500 comprises a mechanical stimulation arrangement 550 that comprises a flexible conductor 531, an actuator 527, a rod 529, and a coupling element 535. Mechanical stimulation arrangement 550 may be similar to stimulation arrangement 150 discussed above with reference to FIGS. 1 and 3.

For ease of explanation, FIG. 5 uses common reference numerals for identifying the components of the recipient's outer ear 101, middle ear 105 and inner ear 107, as used in FIG. 1. These components were each discussed above with reference to FIG. 1 and, as such the discussion is not repeated here.

Cochlear implant 500 comprises an external component 542 which is directly or indirectly attached to the body of the recipient, and an internal component 544 which is temporarily or permanently implanted in the recipient. External component 542 comprises one or more sound input elements, such as microphone 524 for detecting sound, a sound processing unit 526, a power source (not shown), and an external transmitter unit 528. External transmitter unit 528 comprises an external coil 530 and, preferably, a magnet (not shown) secured directly or indirectly to external coil 530. Sound processing unit 526 processes the output of microphone 524 that is positioned, in the depicted embodiment, by auricle 110 of the recipient. Sound processing unit 526 generates encoded signals, sometimes referred to herein as encoded data signals, which are provided to external transmitter unit 528 via a cable (not shown).

Internal component 544 comprises an internal receiver unit 532, a stimulator unit 520, a stimulating lead assembly 518, and mechanical stimulation arrangement 550. Internal receiver unit 532 comprises an internal coil 536, and preferably, a magnet (also not shown) fixed relative to internal coil 536. Internal receiver unit 532 and stimulator unit 520 are hermetically sealed within a biocompatible housing, sometimes collectively referred to as a stimulator/receiver unit. Internal coil 536 receives power and stimulation data from external coil 530, as noted above.

Stimulating lead assembly 518 has a proximal end connected to stimulator unit 520, and a distal end implanted in cochlea 140. Stimulating lead assembly 518 extends from stimulator unit 520 to cochlea 140 through temporal bone 119. In some embodiments stimulating lead assembly 518 may be implanted at least in basal region 116, and sometimes further into cochlea 140. For example, stimulating lead assembly 518 may extend towards apex 134 of cochlea 140. In certain circumstances, stimulating lead assembly 518 may be inserted into cochlea 140 via a cochleostomy 122. In other circumstances, a cochleostomy may be formed through round window 113, oval window 112, the promontory 115 or through an apical turn 147 of cochlea 140. As used herein the term “stimulating lead assembly,” refers to any device capable of providing stimulation to a recipient, such as, for example, electrical or optical stimulation. A such, it should be understood that stimulating lead assembly 518 merely provides one embodiment of an exemplary stimulating lead assembly, and other types of stimulating lead assemblies may be used in other embodiments.

Stimulating lead assembly 518 comprises a longitudinally aligned and distally extending array 546 of electrode contacts 548, sometimes referred to as array of electrode contacts 546 herein, disposed along a length thereof. In most practical applications, array of electrode contacts 546 is integrated into stimulating lead assembly 518. As such, array of electrode contacts 546 is referred to herein as being disposed in stimulating lead assembly 518.

Stimulating lead assembly 118 preferably is positioned in cochlea 140 upon or immediately following implantation into cochlea 140. It is also desirable that stimulating lead assembly 518 be configured such that the insertion process causes minimal trauma to the sensitive structures of cochlea 140. Typically, stimulating lead assembly 518 is pre-curved, held in a substantially straight configuration at least during the initial stages of the implantation procedure, and then permitted to conform to the natural shape of the cochlea during and subsequent to implantation.

Stimulation arrangement 550 comprises an actuator 527, a rod 529, a flexible conductor 531, and a coupling element 535. As illustrated, actuator 527 may be external to stimulator unit 520 and connected to stimulator unit 520 via a cable 537. However, in other arrangements, actuator 527 may be included in a common housing with stimulator unit 520 along with rod 529 and flexible conductor 531 exiting such housing. Actuator 529 may be attached to mastoid bone 119, such as for example, as discussed above with reference to FIG. 2. For example, actuator 529 may be fixed to bone 119 using screws that may be inserted through openings in the actuator 529 and inserted into bone 119. For ease of illustration, ossicles 106 have been omitted from FIG. 5. It should be appreciated that in embodiments flexible conductor 131 and the other components may be implanted without disturbing ossicles 106. Stimulation arrangement 550 may function, for example, in a manner similar to stimulation arrangement 550 discussed above with reference to FIG. 1-4. For example, flexible conductor 531 may include a sheath such as discussed with reference to FIG. 2 and may be coupled to coupling elements such as discussed with reference to FIG. 4.

In operation, sound is received by external component 542, processed, encoded, and transmitted to stimulator unit 520 using external transmitter unit 528. External coil 530 of external transmitter unit 528 may transmit electrical signals (i.e., power and stimulation data) to internal coil 536 via a radio frequency (RF) link. Internal coil 536 may be a wire antenna coil comprised of multiple turns of electrically insulated single-strand or multi-strand platinum or gold wire. The electrical insulation of internal coil 536 may be provided by a flexible silicone molding (not shown). In use, implantable receiver unit 532 may be positioned in a recess of the temporal bone adjacent auricle 110 of the recipient.

Internal receive unit 532 provides the received electrical signals to sound processing unit 526. Sound processing unit 526 may then process the received data signals and generate corresponding stimulation signals for both electronically and mechanically stimulating the auditory nerve of the recipient. The generated stimulation signals are then transferred to stimulator unit 520 which then provides corresponding stimulation signals to actuator 527 and/or stimulating lead assembly 518. Stimulator unit 520 may, for example, comprise a stimulator for generating electrical stimulation signals for applying electrical stimulation using electrode contacts 548 of stimulating lead assembly 518.

In an embodiment, cochlear implant 500 is configured to provide electrical stimulation for higher (i.e., frequencies above a threshold frequency) and mechanical stimulation for lower frequencies (i.e., frequencies below a threshold frequency). In such an embodiment, sound processing unit 526 may filter the received signal data signals (i.e., the electrical signals representative of the sound received by the microphone) to split the received signal into a higher frequency signal and a low frequency signal. This may be accomplished, for example, by splitting the signal into two signals and applying a low pass filter to one signal and a high pass filter to the other signal.

After splitting the received signal into a high frequency signal and a low frequency signal, sound processing unit may independently process each signal. For example, sound processing unit 526 may process the high frequency signal to generate a stimulation signal for application of electrical stimulation using stimulating lead assembly 518.

Sound processing unit 526 may process the low frequency signal to generate a stimulation signal for use by actuator 527. Sound processing unit 526 may then transmit each of these stimulation signals to stimulator unit 520. Stimulator unit 520 may then apply electrical stimulation via stimulating lead assembly 518 in accordance with the received electrical stimulation signal. Stimulator unit 520 may also simultaneously cause actuation of actuator 527 in accordance with the received mechanical stimulation signal.

Because in this example, cochlear implant 500 only applies higher frequency signals using stimulating lead assembly, cochlear implant 500 may use a shorter stimulating lead assembly than would be typically be used in a non-mixed mode device in which the stimulating lead assembly is to be used for applying electrical stimulation for frequencies across the normal hearing range (e.g., approximately 20-20 kHz). This shorter stimulating lead assembly may only be inserted into the basal region of cochlea 140 so as to not damage the hairs located in the more apical portion of cochlea 140. Thus, the mechanical movements of the perilymph induced by the mechanical movements of mechanical stimulation arrange 550 may cause activation of the tiny hair cells located in these more apical portions of cochlea 140. As noted above, activation of the hair cells causes appropriate nerve impulses to be generated and transferred through the spiral ganglion cells (not shown) and auditory nerve 114 to the brain (also not shown) where they are perceived as sound.

In another embodiment, rather than stimulating lead assembly 518 being directly connected to stimulator unit 520, the stimulating lead assembly may be connected to the stimulator by a flexible connector. This flexible connector may include a wire for transporting mechanical energy from an actuator as well as a wire(s) (e.g., leads) for carrying electrical stimulation signals for application of stimulation using the electrode contacts of the stimulating lead assembly. The two wires may then split in the middle ear or inner ear such that the wire for mechanical stimulation is coupled to a component of the middle or inner ear of the recipient. Or, for example, in an embodiment, the wire for mechanical stimulation may travel at least partially through the stimulating lead assembly and exit the stimulating lead assembly such that when implanted the wire will be located within cochlea 140. The end of the wire may comprise a coupling element (e.g., a rectangular, circular, ovular, or square plate) for helping transfer vibrations, during operation, from the actuator to the perilymph of the cochlea.

Although the embodiments of FIGS. 1-5 have been described with reference to hearing prostheses having an external component, it should be appreciated that in alternative embodiments the hearing prosthesis may be a totally implantable device. In such embodiments, the sound processing unit may be, for example, implanted in a recess in the mastoid bone of the recipient.

In an embodiment, a flexible conductor such as discussed above may be surgically implanted in a recipient. FIG. 6 illustrates a simplified flow chart illustrating operations for surgically inserting a flexible conductor within a patient. FIG. 6 will be discussed with reference to implantation of a flexible conductor such as discussed above with reference to FIG. 1

Flexible conductor 131 may be initially connected to a coupling element 135 at block 602. This may be accomplished, for example, prior to surgery. However, in other embodiments, this connecting may be accomplished at some other point, such as in the middle of the surgical procedure. The type of coupling element 135 may be selected depending on, for example, the particulars of the type of stimulation to be delivered to the patient. For example, a coupling element such as discussed above with reference to FIG. 4 may be used, or some other type of coupling element may be connected to flexible conductor 131.

An opening in the mastoid bone may be surgically created at block 604. Then, flexible conductor 131 may be inserted through the opening at block 606. The coupling element 135 may then be coupled to an interior component of the recipient's ear at block 608. In the embodiment of FIG. 1, the coupling element 135 is coupled to oval window 112. However, in other embodiments, coupling element 135 may be coupled to other inner ear components, such as a semicircular canal 125 or round window 113. Or, in yet other embodiments, a fenestration may be surgically created in the bony structure of cochlea 140 and the flexible conductor 131 inserted through the fenestration. This fenestration may be, for example, sealed using fibrous tissue to prevent perilymph from leaking out of cochlea 140 after insertion of flexible conductor 131 though the fenestration. Flexible conductor 131 may then be fixed to a structure within the recipient at block 610 to prevent or limit movement of the flexible conductor 131, such as was discussed above with reference to FIG. 3.

Flexible conductor 131 may then be coupled at block 612 to rod 129 coupled to actuator 131. As noted above, actuator 127 may be inserted into and fixed in place in a recess created in the mastoid bone 119 of the recipient. During blocks 606-612, the surgeon may manually bend flexible conductor 131 as appropriate. After block 612, other components, if any, may be implanted within the recipient. For example, if the hearing prosthesis is a mixed-mode device addition components, such as stimulating lead assembly may be inserted into recipient. Or, for example, certain of these components may be implanted prior to connection of the flexible conductor and the coupling element and/or rod.

It should be noted that the flow chart of FIG. 6 is but one simplified process for implanting a flexible conductor and other methods may be used. Further, the order of the steps of FIG. 6 is exemplary only and in other embodiments a different ordering of steps may be used.

Referring back to FIG. 1B, in an embodiment, actuator 127 (or another component, such as sound processing unit 126) may compensate for deviations in an acoustic transfer function of the stimulation arrangement 150. For example, in an embodiment, the acoustic transfer function (also sometimes referred to as the sound transfer function) of the stimulation arrangement 150 comprising the actuator 127, rod 129, flexible conductor 131 and the coupling element 135 may be predictable only in a certain range due to anatomic imponderability and device variations. In particular, the acoustic transfer function may depend, among other parameters, on the length of the coupling element 135, the thickness of the coupling element 135, the acoustic impedance of the coupling element 135 and its surrounding material, and on the path on which the coupling element 135 is arranged in the ear.

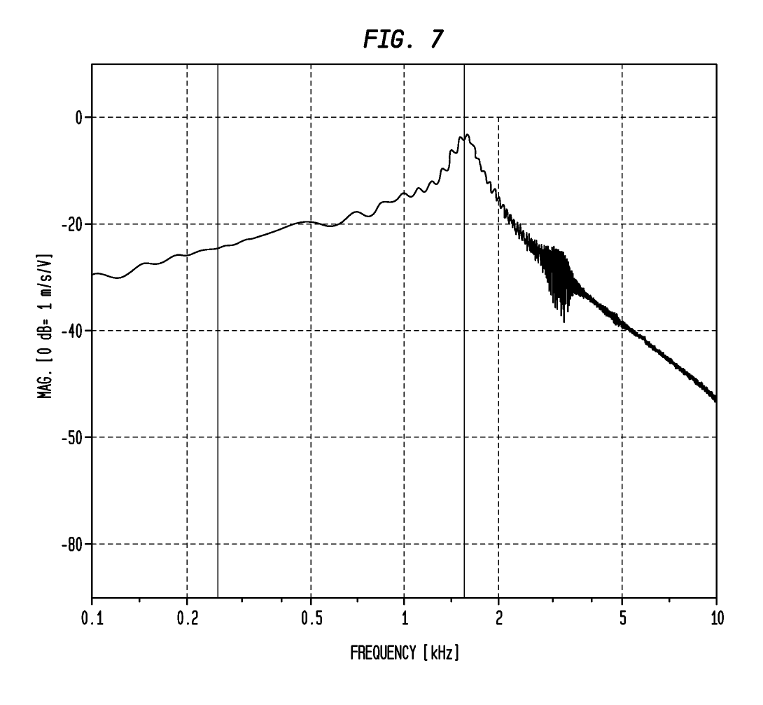

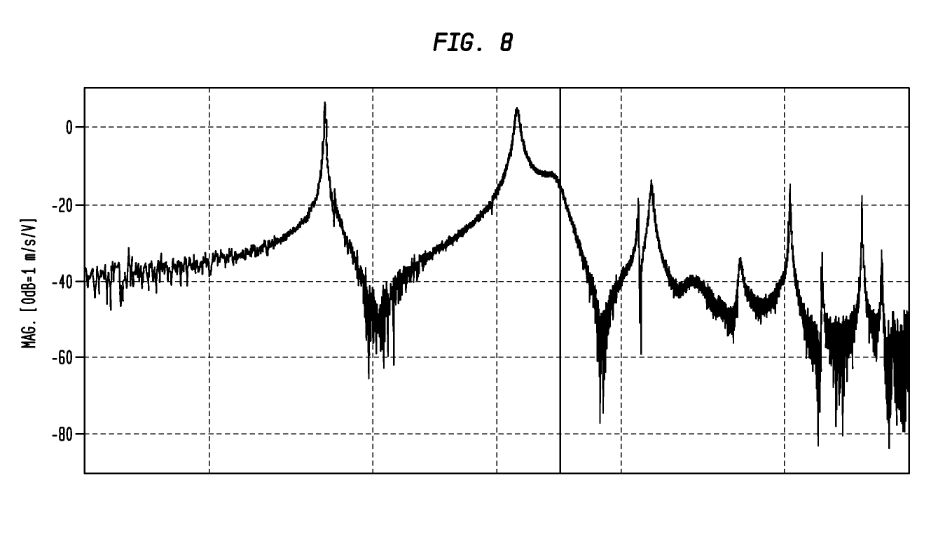

FIG. 7 illustrates an exemplary acoustic transfer function of a specific DACS actuator using a stiff connection between the actuator and a structure in the middle ear or inner ear. As illustrated, the acoustic transfer function of this device shows a single resonance structure and is rather predictable and is used as a acoustic transfer function.

FIG. 8 illustrates an exemplary transfer function of a stimulation arrangement including a DACS actuator and a flexible sound conductor. The measurement conditions for FIG. 8 comprised the usage of a flexible sound conductor having a length of 2.3 cm that was attached to a rod of the transducer. 0.3 cm of the flexible sound conductor overlapped with the rod, i.e. 2 cm of the flexible sound conductor 100 was free hanging. The total weight of the flexible sound conductor was 11,285 mg. The flexible sound conductor had an open end, free hanging in the air. The opposite end of the flexible sound conductor, which was connected to the rod, was bent into a horizontal direction by 90° degree.

As seen in FIG. 8, the acoustic transfer function in this example deviates from the standard transfer function illustrated in FIG. 7. On the other hand, FIG. 8 clearly indicates that sound is transmitted using the exemplary stimulation arrangement.

In order to achieve as transmission properties similar to the standard sound transfer function (FIG. 7), a actuator 127 (or another component, such as sound processor 126) may comprises software and/or hardware for compensating for deviations of the acoustic transfer function from a desired sound transfer function. For example, actuator 127 (or another component) may comprise one or more filters that compensate for the acoustic transfer function. This compensation may designed so that the net acoustic transfer function of the stimulation arrangement 150 has a response matching or similar to the sound transfer function of FIG. 7, has a flat response, or has some other shape.

The present disclosure also includes according to another aspect the provision of a sound transmission device comprising an implantable actuator adapted for generating energy representing sound, and a flexible sound conductor coupled at a first end thereof to the implantable actuator and being configured for transmitting sound from the actuator to a structure in the middle ear or the inner ear. The sound transmission device may be adapted to use mechanical, acoustical, optical, and/or electromechanical energy as the energy representing sound. An actuation principle may be one of piezoelectric and electromagnetic. The sound transmission device according to this aspect may further comprise a device for compensating deviations of a sound transfer function due to the flexible sound conductor.

It is to be understood that the detailed description and specific examples, while indicating embodiments of the present invention, are given by way of illustration and not limitation. Many changes and modifications within the scope of the present invention may be made without departing from the spirit thereof, and the invention includes all such modifications.

What is claimed is:

1. A hearing prosthesis for delivering sound vibrations to a component of a recipient's ear, the hearing prosthesis comprising:
an implantable actuator configured to generate the sound vibrations; and
a longitudinally-rigid and laterally-flexible elongate conductor adapted to be connected to the actuator and adapted to be coupled to the ear component, wherein the flexible conductor is adapted to transport the sound vibrations from the actuator to the interior component of the ear.

2. The hearing prosthesis of claim 1, wherein the ear component is a one either a middle ear component and an inner ear component

3. The hearing prosthesis of claim 2, wherein the ear component is a bone of the ossicular chain.

4. The hearing prosthesis of claim 2, wherein the ear component is one of the group consisting of:
the cochlear promontory;
a natural fenestration of the cochlea; and
an artificial fenestration of the cochlea or semicircular canals.

5. The hearing prosthesis of claim 1, wherein the flexible conductor is configured to have an impedance mismatch with surrounding tissues when implanted in the recipient.

6. The hearing prosthesis of claim 1, wherein the flexible conductor is adapted to transfer said vibrations from the actuator to the ear component so as to generate movement of perilymph located in a cochlea of the recipient.

7. The hearing prosthesis of claim 4, further comprising:
a coupling element connected to the flexible conductor and configured to couple the flexible conductor to the ear component.

8. The hearing prosthesis of claim 1, wherein the actuator is configured to be implanted in a recess of a mastoid bone of the recipient.

9. The hearing prosthesis of claim 1, wherein the flexible conductor comprises a wire.

10. The hearing prosthesis of claim 9, wherein the flexible conductor comprises:
a wire; and
a sheath disposed over the wire, the sheath adapted to acoustically decouple the wire, when implanted in the recipient, from tissue interior to the recipient and proximal to the flexible conductor.

11. The hearing prosthesis of claim 11, wherein the sheath comprises a silicone sleeve.

12. The hearing prosthesis of claim 1, further comprising:
a stimulating lead assembly adapted to be implanted at least partially within a cochlea of the recipient and comprising one or more electrode contacts; and
a stimulator unit configured to deliver a stimulation signal to an electrode contact for applying electrical stimulation to the cochlea in accordance with the acoustic signal.

13. A method comprising:
implanting a flexible conductor in a recipient, wherein the flexible conductor is longitudinally rigid and laterally flexible;
coupling a first end of the flexible conductor to a component of an ear of the recipient; and
coupling a second end of the flexible conductor to an actuator configured to generate sound vibrations representative of an acoustic signal, such that the flexible conductor is configured to transport energy from the energy source to the ear component

14. The method of claim 13, further comprising:
receiving the acoustic signal;
generating, by the actuator, vibrations representative of the acoustic signal; and
transferring, by the flexible conductor, said vibrations from the actuator to the interior component of the ear of the recipient so as to generate movement of perilymph located in a cochlea of the recipient.

15. The method of claim 14, wherein coupling the first end of the flexible conductor comprises:
connecting the first end of the flexible conductor to a coupling element; and
coupling the coupling element to the ear component.

16. The method of claim 15, wherein coupling the coupling element to the interior component comprises:
coupling the coupling element to a component of a middle ear of the recipient;
wherein the ear component is a bone of the ossicular chain.

17. The method of claim 15, wherein coupling the coupling element to the ear component comprises:
coupling the coupling element to a component of an inner ear of the recipient selected from the set of the cochlear promontory; a natural fenestration of the cochlea; and an artificial fenestration of the cochlea or semicircular canals.

18. The method of claim 13, further comprising:
surgically creating a recess in a mastoid bone of the recipient; and
securing the actuator is said recess.

19. The method of claim 13, wherein the flexible conductor comprises a wire.

20. The method claim 13, wherein the flexible conductor comprises:
a wire; and
a sheath disposed over the wire, the sheath adapted to acoustically decouple the wire, when implanted in the recipient, from tissue interior to the recipient and proximal to the flexible conductor.

21. The method of claim 13, further comprising:
surgically implanting a stimulating lead assembly comprising one or more electrode contacts at least partially within a cochlea of the recipient; and
connecting the stimulating lead assembly to a stimulator unit configured to deliver a stimulation signal to an electrode contact for applying electrical stimulation to the cochlea in accordance with the acoustic signal.
